(19) United States
(12) Patent Application Publication
(10) Pub. No.: US 2004/0127512 A1
(43) Pub. Date: Jul. 1, 2004

(54) Method of treating symptoms of non-constipated irritable bowel syndrome

(75) Inventors: Bradley P. Glassman, Fairfield, NJ (US); Dileep Bhagwat, Bronxville, NY (US); Daniel Glassman, Fairfield, NJ (US)

Correspondence Address: COVINGTON & BURLING, ATTN: PATENT DOCKETING, 1201 PENNSYLVANIA AVENUE, N.W., WASHINGTON, DC, 20004-2401, US

(73) Assignee: Bradley Pharmaceuticals, Inc.

(21) Appl. No.: 10/334,170

(22) Filed: Dec. 30, 2002

(51) Int. Cl. A61K 31/4745

(52) U.S. Cl. 514/291

(57) ABSTRACT

Described is a method of treating the symptoms of non-constipated irritable bowel syndrome (IBS) and diarrhea-predominant IBS with methscopolamine bromide.

### FIELD OF THE INVENTION

The present invention relates to a method of treating the symptoms of non-constipated irritable bowel syndrome, such as abdominal pain, cramping, bloating, diarrhea and other quality of life symptoms.

### BACKGROUND OF THE INVENTION

Irritable bowel syndrome (IBS) is the most common diagnosis made by gastroenterologists and is characterized by abdominal pain and discomfort and altered bowel functions. To date, no laboratory tests or other diagnosis have been identified in IBS and the formal diagnosis is based upon a variety of symptoms.

The current understanding of the pathophysiology or etiology of IBS is limited. Moreover, many patients gain slight or even no relief from such therapies. Thus, there is a real need to develop new medicines for the treatment of IBS, or even to relieve the symptoms of IBS. Threshold for sensation of pain is lower in IBS patients compared to controls.

In view of the evidence for hypersensitive visceral perception in IBS and the frequent occurrence of pain, any agent considered to be of utility in the treatment of IBS should demonstrate effectiveness in the relief of pain and other symptoms.

Irritable bowel syndrome (IBS) is one of the functional gastrointestinal disorders attributed to the colon and rectum. The primary symptoms in IBS are chronic abdominal pain or discomfort associated with defecation and/or an alteration in bowel habit. Clinical (symptom-based) diagnosis plus a limited screen for organic disease has been proved to be a safe diagnostic strategy.

In Western countries it is estimated that between 14 and 24% of women and between 5 and 19% of men have IBS symptoms. General trends in study data indicate that IBS symptoms are more prevalent in females than in males and that the prevalence decreases above the age of 60 years. The natural history of IBS is one of waxing and waning. Studies of the same population over time at 12 month or 5 year intervals have shown a constant prevalence, but half the cases identified at the second study interval are new cases. The presence of IBS symptoms is associated with poorer quality of life and increased work absenteeism. Despite the fact that only a third of people with IBS symptoms seek medical attention, patients with IBS “consume” 12% of primary care consultations and 28% of gastroenterology consultations. Thus, the total direct medical and indirect societal costs of IBS are substantial. When the excess direct charges (patient care, prescriptions, hospitalizations, and so forth) calculated for persons with IBS versus non-IBS controls are extrapolated to the entire U.S. population, IBS results in an annual expenditure of over $8 billion.

IBS is a complex biopsychosocial disorder with abnormal mechanisms in brain-gut interactions. Abnormal motor functions and heightened visceral sensitivity are factors in origin of symptoms of altered bowel function and pain. Abnormal colonic motility or sensation is clearly modulated by input from the central nervous system, including the higher centers. Psychological distress alters bowel habits and leads to abdominal pain or discomfort in healthy persons. IBS is characterized by increased gut motility and sensory responses to psychologic or physiologic stressors. Gastrointestinal motor patterns in IBS are perceived as being painful in IBS patients, but not in healthy control subjects. The defect may be at one or more levels of the sensory apparatus, from the primary sensory receptors of the bowel to the brain centers perceiving visceral pain.

The most crucial step in treatment is correct diagnosis and reassurance to avoid further unnecessary tests, surgery, and “doctor shopping”. Differentiation of IBS from other functional gastrointestinal disorders, especially constipation secondary to evacuation disorders, is necessary.

The intensity of IBS symptoms is highly variable. Therapy must therefore be highly individualized and directed at the most bothersome symptom.

Although some patients focus a great deal on the relationship of symptoms to specific foods, there is no convincing evidence linking diet to IBS.

### SUMMARY OF THE INVENTION

This invention relates to a new medical use for the compound methscopolamine bromide which includes relieving the symptoms of irritable bowel syndrome like abdominal pain, cramping, bloating and diarrhea.

Surprisingly, it has now been found that methscopolamine bromide is a particularly effective and well tolerated therapy in treating the symptoms of irritable bowel syndrome (IBS) in patients suffering therefrom.

According to one aspect the invention provides administering an effective amount of methscopolamine bromide and one or more pharmaceutically acceptable carriers or excipients for use in treating the symptoms of non-constipated IBS patients.

In another aspect the invention provides administering an effective amount of methscopolamine bromide and one or more pharmaceutically acceptable carriers or excipients for use in treating the symptoms of diarrhea predominant IBS patients.

### DETAILED DESCRIPTION OF THE INVENTION

Methscopolamine bromide, an anticholinergic agent, occurs as white crystals, or as a white odorless crystalline powder. Methscopolamine bromide melts at about 225° C. with decomposition. The drug is freely soluble in water, slightly soluble in alcohol, and insoluble in acetone and in chloroform.

The chemical name for methscopolamine bromide is [7(S)-(1α, 2β, 5α, 7β]-7-(3-hydroxy-1-oxo-2-phenylpropoxy)-9,9-dimethyl-3-oxa-9-azoniatricyclo [3.3.1.0²,⁴] nonane, and the molecular weight is 398.31. The compound is prepared by the action of methyl bromide on scopolamine base as described in U.S. Pat. No. 2,753,288, which patent is incorporated herein by reference.

Methscopolamine bromide is an anticholinergic agent which possesses most of the pharmacologic actions of that drug class. These include reduction in volume and total acid content of gastric secretion, inhibition of gastrointestinal motility, inhibition of salivary excretion, and dilation of the pupil. The compound has been used in the treatment of duodenal ulcer by transdermal administration, R. P. Walt et al., Brit. Med. J. 7-84, 1736 (1982).

Methscopolamine bromide is a quaternary ammonium derivative of scopolamine. Quaternary ammonium salts have limited absorption from intact skin, and conjunctival penetration is poor. Little is known of the fate and excretion of most of these agents. Following oral administration, drug effects appear in about one hour and persist for 4 to 6 hours. Methscopolamine bromide has limited ability to cross the blood-brain-barrier. The drug is excreted primarily in the urine and bile, or as unabsorbed drug in feces. There is no data on the presence of methscopolamine in breast milk. Currently this drug is indicated for the adjunctive therapy for the treatment of peptic ulcer.

The invention provides methscopolamine bromide in a unit dosage form containing one or more pharmaceutically acceptable carriers or excipients for use in the treatment of symptoms from IBS.

Accordingly, the invention provides a method of treatment of IBS which comprises administering an effective amount of methscopolamine bromide.

It is to be understood that reference to treatment includes both treatment of established symptoms and prophylactic treatment, unless explicitly stated otherwise.

Conveniently, methscopolamine bromide may be formulated in a conventional manner using one or more pharmaceutically acceptable carriers or excipients. Thus methscopolamine bromide may, for example, be formulated for oral, sublingual, buccal, parenteral, rectal or intranasal administration, or in a form suitable for administration by inhalation or insufflation (either through the mouth or nose), or in a form suitable for topical administration.

For oral administration the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium phosphate); lubricants (e.g. magnesium stearate, talc or silica); disintegrates (e.g. potato starch or sodium starch glycolate); or wetting agents (e.g. sodium lauryl sulphate). The tablets may be coated by methods well known in the art.

Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl-p-hydroxybenzoates or sorbic acid).

For buccal administration the compositions may take the form of tablets or lozenges formulated in a conventional manner.

For parenteral administration the compositions may take the form of injections, conveniently intravenous, intramuscular or subcutaneous injections, for example bolus injections or continuous intravenous infusions. Formulations for injection may be presented in unit dosage form e.g. in ampoules or in multi-dose containers, optionally with an added preservative.

The compositions for parenteral administration may take such forms as suspensions, solutions or emulsions in oily or aqueous vehicles, and may contain formulatory agents such as suspending, stabilizing and/or dispersing agents. Alternatively, the compositions may be in dry form such as a powder, crystalline or freeze-dried solid for constitution with a suitable vehicle, e.g. sterile pyrogen-free water or isotonic saline before use. They may be presented, for example, in sterile ampoules or vials.

For rectal administration the compositions may take the form of suppositories or retention enemas.

Tablets for sub-lingual administration may be formulated in a conventional manner.

For intranasal administration or administration by inhalation or insufflation, conventional formulations may be employed.

For topical administration the pharmaceutical compositions may be liquids, for example solutions, suspensions or emulsions presented in the form of creams or gels or transdermal patch.

In addition to the formulations described previously, the compositions may also be formulated as depot preparations. Such long acting formulations may be administered by implantation (for example subcutaneously, transcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

It will be appreciated that the precise therapeutic dose of methscopolamine bromide will depend on the age and condition of the patient and the nature of the IBS to be treated, and will be at the ultimate discretion of the attendant physician.

However, in general, effective doses for the treatment of nonconstipated IBS patients will lie in the range of 0.001 to 50 mg, such as 0.01 to 50 mg, preferably 2.5 to 50 mg, for example 0.5 to 50 mg per unit dose, which could be administered in single or divided doses, for example 1 to 4 times per day.

In a preferred embodiment, effective doses of methscopolamine bromide for the treatment of diarrhea predominant IBS patients will lie in the range of 0.01 to 100 mg, such as 0.05 to 50 mg, preferably 0.1 to 25 mg, for example 2.5, 5, 10, 20 mg of methscopolamine per unit dose, which could be administered in single or divided doses, for example, 1 to 4 times per day.

### EXAMPLE

A clinical study was undertaken with oral methscopolamine bromide as an antispasmodic medication in colonoscopy. The study comprised of 40 non-randomized patients. Thirty patients were given oral methscopolamine bromide prior to the procedure. As a control population 10 patients received no oral methscopolamine bromide. The first 5 patients took 5 mg of oral methscopolamine bromide one hour before colonoscopy. The dose was increased to 10 mg for the following 25 patients. For the purpose of the study, in terms of spasm control a “success” was defined as a colonoscopy where there was no significant spasm on insertion and no intravenous Buscopan® was required. A failure was defined as the requirement of intravenous Buscopan® (antispasmodic). Overall 64% of patients taking 10 mg of oral methscopolamine bromide had minimal or no spasm as compared with only 30% of control subjects. The study had both male and female patients.

What is claimed is:

1. A method of treating the symptoms of a non-constipated IBS patient which comprises administering to said patient suffering therefrom an effective amount of methscopolamine bromide.

2. The method of claim 1 wherein methscopolamine bromide is in admixture with one or more pharmaceutically acceptable carriers or excipients.

3. The method of claim 2 wherein methscopolamine bromide is administered orally.

4. A method of treating the symptoms of a diarrhea-predominant IBS patient which comprises administering to said patient suffering therefrom an effective amount of methscopolamine bromide.

5. The method of claim 4, wherein methscopolamine bromide is in admixture with one or more pharmaceutically acceptable carriers or excipients.

6. The method of claim 5, wherein methscopolamine bromide is administered orally.
